(19) United States
(12) Patent Application Publication
(10) Pub. No.: US 2006/0020269 A1
(43) Pub. Date: Jan. 26, 2006

(54) Device to aid in stone removal and laser lithotripsy

(76) Inventor: Eric Cheng, Bloomington, IN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/894,122

(22) Filed: Jul. 20, 2004

(51) Int. Cl. A61B 17/26

(52) U.S. Cl. 606/113

(57) ABSTRACT

A medical device includes a delivery tube and a sponge configured for deployment from the delivery tube and into the urinary tract for assisting in the removal of a stone.

### FIELD OF THE INVENTION

This device relates generally to medical devices and procedures and more particularly to medical devices and procedures for assisting in the removal of stones from a body.

### BACKGROUND OF THE INVENTION

Extraction devices have been used for the removal of stones or calculi from the urinary tract of the human body. Typically, the distal portion of existing devices includes wires or strips that can be manipulated inside the body to expand, forming an open basket. Through further manipulation, the targeted stone can be captured within the basket. The device is then withdrawn from the body, thereby removing the stone.

The baskets of existing devices are rigid as compared to the surrounding body tissue. Due to their rigidity, these devices can cause harm to the body tissue or body structure in which stones reside. For example, existing devices may not be flexible enough to conform to the contours of the treatment location during capture or removal of the stone. As a result, manipulation of the device in the body and removal of a stone residing in, for example, the ureter may damage the ureter wall. Such damage to the ureter wall may cause various trauma to the wall, necessitating the placement of a temporary stent within the ureter.

Existing devices may also use lasers or pneumatics to diminish the size of the stone before removing it. While being acted on by these devices, however, parts of the stone may break free and migrate to other parts of the body. They may also migrate to calyxes of the same body structure in which the stone resides.

The present disclosure provides a device that assists in stone removal that avoids some or all of the aforementioned shortcomings of existing devices.

### SUMMARY OF THE INVENTION

In accordance with one aspect of the present disclosure, a medical device includes a delivery tube and a sponge configured for deployment from the delivery tube and into the urinary tract for assisting in the removal of a stone.

In accordance with another aspect of the present disclosure, a method for assisting the removal of a stone from a body includes deploying a sponge into the body and contacting the stone with the sponge to facilitate removal of the stone from the body.

In accordance with yet another aspect of the present disclosure, a method for assisting the removal of a stone from a body includes deploying a sponge into the body and prohibiting the stone from migrating distal the sponge while the stone is subject to a procedure.

It is to be understood that both the foregoing general description and the following detailed description are exemplary and explanatory only, and are not restrictive of the invention as claimed.

### BRIEF DESCRIPTION OF THE DRAWINGS

FIG. 1 is a cross-sectional view of a stone removal assistance device according to an exemplary embodiment of the present disclosure;

FIG. 2 is a cross-sectional view of the device of FIG. 1 positioned within the body;

FIG. 3 is an operational view of the device of FIG. 1;

FIG. 4 is another operational view of the device of FIG. 1;

FIG. 5 is a cross-sectional view of a sponge according to an exemplary embodiment of the present disclosure;

FIG. 6 is a cross-sectional view of a sponge according to another exemplary embodiment of the present disclosure;

FIG. 7 is a cross-sectional view of a sponge according to still another exemplary embodiment of the present disclosure;

FIG. 8 is a cross-sectional view of a sponge according to yet another exemplary embodiment of the present disclosure; and

FIG. 9 is a further operational view of the device of FIG. 1.

### DETAILED DESCRIPTION

Exemplary embodiments of the present disclosure are illustrated in the accompanying drawings. Wherever possible, the same reference numbers will be used throughout the drawings to refer to the same or like parts.

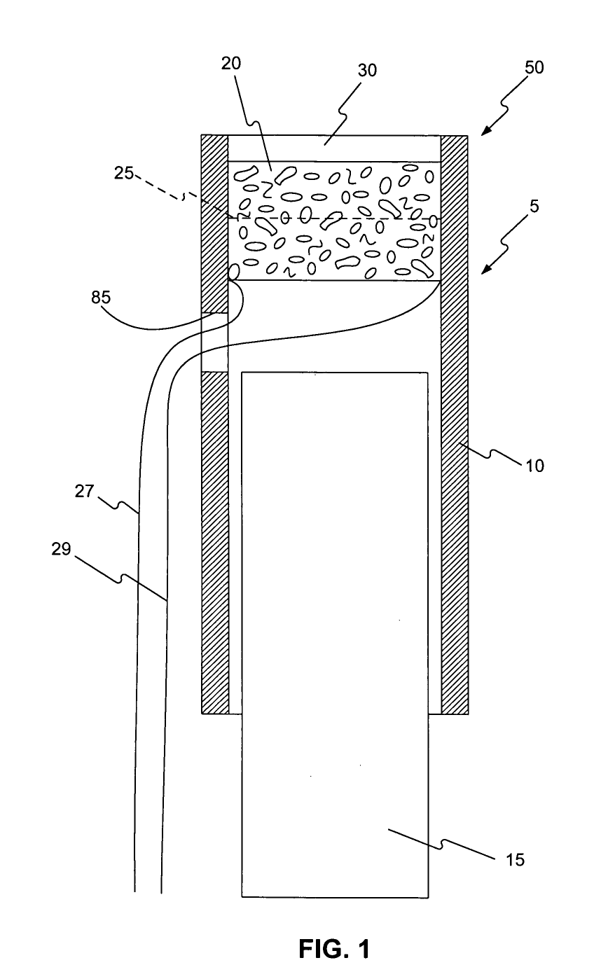

FIG. 1 illustrates a device 5 that assists in stone removal according to an embodiment of the present disclosure. The device 5 may include a delivery tube 10, an urging member 15, and a sponge 20 having at least one tethering member 25 extending through the sponge 20 and forming a first and second leg 27, 29.

As shown in FIG. 1, the delivery tube 10 may provide a channel 30 within which the urging member 15 and the sponge 20 may move. The delivery tube 10 may be constructed of tubing commonly known in the art. Such tubing materials may include, but are not limited to, stainless steel or nitinol. They may also include plastics, composites, or polymers, such as urethanes, Teflon, polyimide, or nylons, depending on the desired rigidity or flexibility of the delivery tube 10. Braiding or webbing made from these same materials could also be added to the delivery tube 10 to achieve a desired rigidity.

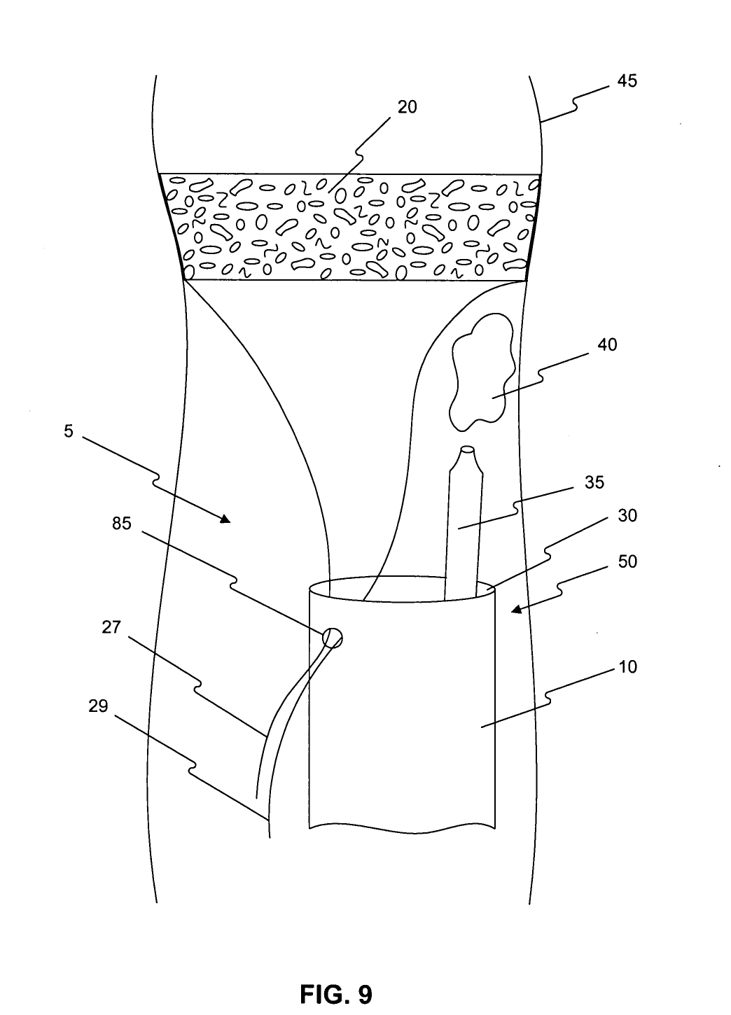

The delivery tube 10 or delivery member may be a tubular member, and may be cylindrical or some other like shape to facilitate easy insertion into the body, and easy manipulation once inside of the body. The delivery tube 10 may be hollow and may be relatively flexible as compared to the urging member 15. The channel 30 of the delivery tube 10 may also provide one or more additional passages within which other medical devices such as, but not limited to, laser fibers 35 (FIG. 9) used in intracorporeal laser lithotripsy may travel.

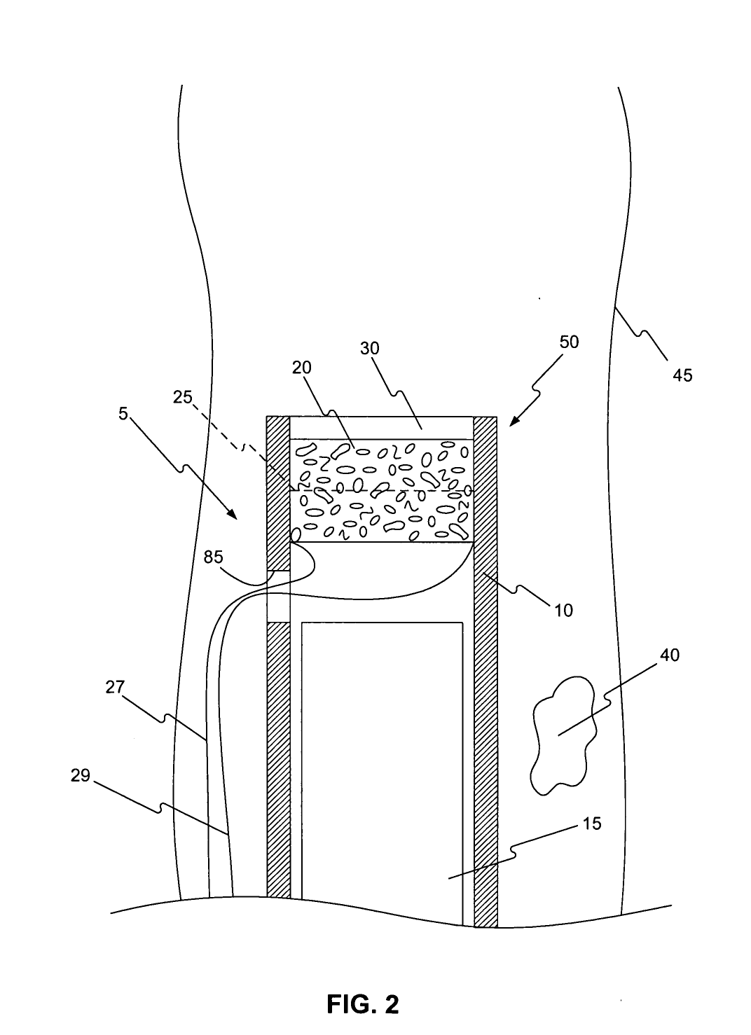

The sponge 20 may be any material that expands when coming into contact with a fluid. For example, the sponge 20 may be a polyvinyl alcohol sponge, or other sponge-like material commonly known in the art. As shown in FIG. 2, in its unexpanded state, the sponge 20 may be compressed to fit within the channel 30 of the delivery tube 10. To minimize the size of the device 5, it may be desirable for the delivery tube 10 to be as small as possible thereby limiting the size of the sponge 20 in its unexpanded state. The sponge 20 may be compressed to fit within the channel 30 by any means possible. For example, the sponge 20 may be compressed through radial compression in which it is twisted clockwise or counter-clockwise relative to the delivery tube 10 while being inserted therein. The sponge 20 may also be folded or pleated for insertion into the delivery tube 10. As a further example, the sponge 20 may be compressed for insertion by pressing a rod or other implement on two or more of its sides.

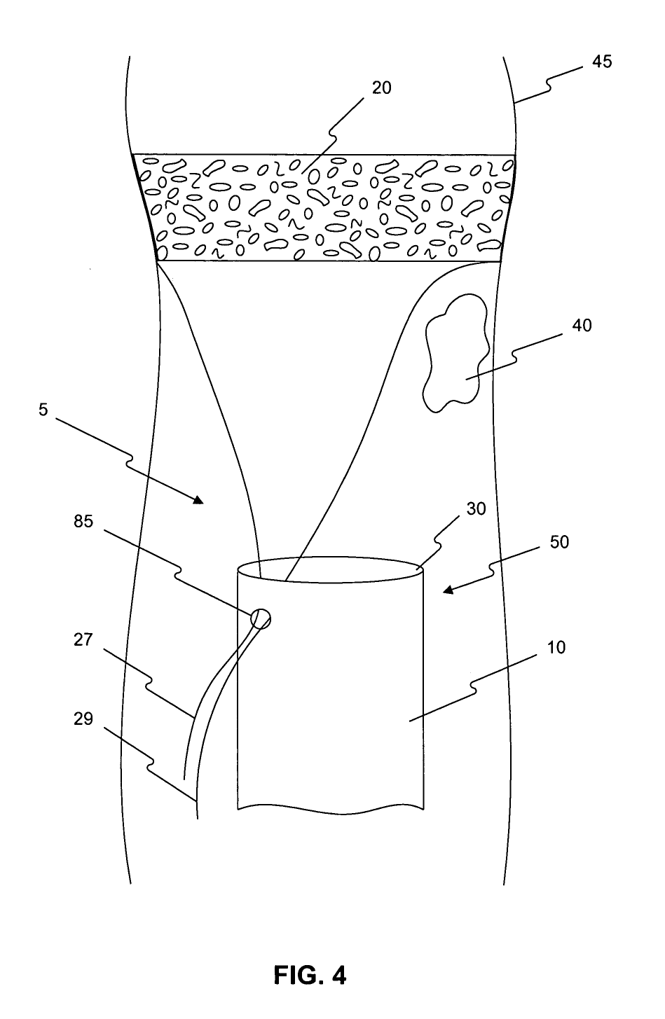

On the other hand, the sponge 20 may be of any size in its expanded state and, as shown in FIG. 4, may be sized to extend fully across the ureter or other body structure of the patient. The sponge 20 may be slightly oversized relative to the body structure to impede the passage of material between the sponge 20 and the body tissue 45 (FIG. 4). In its expanded state, the sponge 20 may have a diameter of approximately 2 mm to 10 mm, and may have a compression ratio (expanded state to unexpanded state) of approximately 10 to 1. It is understood that these diameters and ratios are merely exemplary and that the device 5 of the present disclosure may utilize sponges 20 that fall outside of these ranges.

The sponge 20 may be of any shape or design to facilitate the immobilization and removal of stones within the urinary tract. For example, the sponge 20 may form a cylindrical or drum-like shape in an expanded state as shown in FIG. 4. These shapes or designs may correspond to the body structure in which a stone 40 resides. They may also be designed to facilitate the compression of the sponge 20 and assist in stone retrieval and immobilization. For example, although not shown, a side of the sponge 20 may be concave. This shape may enable the user to compress the sponge 20 with less force or effort and may improve the immobilization and retrieval characteristics of the sponge 20 while in its expanded state.

In addition, the sponge 20 may also be shaped to facilitate re-entry of the sponge 20 into the delivery tube 10. For example, a side of the sponge 20 may be angled or tapered to ease entry of the sponge 20 into the channel 30 of the delivery tube 10 after the sponge 20 has absorbed fluid and is in its expanded state. To accomplish re-entry, the sponge 20 may release at least some of the fluid absorbed.

Referring to FIG. 2, the stone 40 may be a kidney stone, a struvite, a uric acid stone, a cystine stone, or other solid deposit commonly removed from a body structure or passageway within the body. Such stones 40 may contain various combinations of chemicals including, but not limited to, calcium, oxalate, and phosphate. The stone 40 may be of any size, and could have a length or diameter of approximately 1 mm to 12 mm. It is understood that these lengths and diameters are merely exemplary and that aspects of the present disclosure may assist in the removal of stones 40 larger or smaller than those discussed herein.

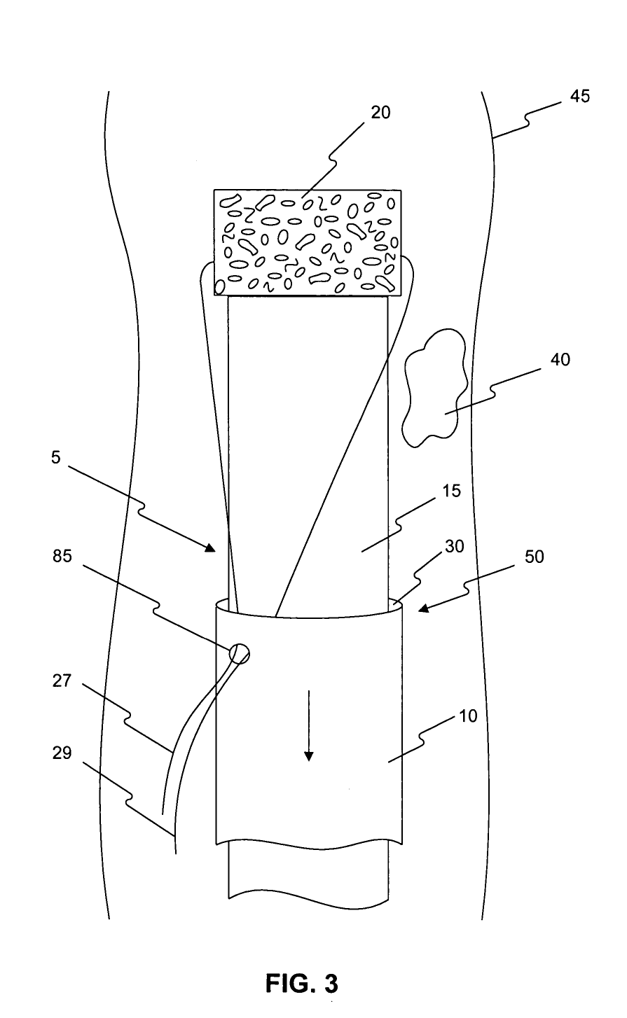

It is further understood that stones 40 may be of any shape, and could be, for example, flat, round, smooth, or jagged. Although FIGS. 2-4 and FIG. 9 show a stone 40 in close proximity to body tissue 45, the device 5 may assist in the immobilization and removal of stones 40 that are both impacted and free floating.

As illustrated in FIGS. 2-4, the sponge 20 may be urged from the distal end 50 of the delivery tube 10 by the urging member 15. As used herein, the term “distal” means furthest from the user. The urging member 15 may be a solid rod or tube, and may be constructed from any material commonly known in the art including, but not limited to, stainless steel or nitinol mandrel tubing, or polymers, such as urethanes or nylons. The urging member 15 may be of a size and shape to fit within the delivery tube 10. The urging member 15 may also be of a size, shape, and rigidity to urge the sponge 20 to exit from the distal end 50 of the delivery tube 10 once the device 5 has been delivered to a desired location within the urinary tract.

As noted above, the sponge 20 may be secured to the delivery tube 10 by a coupling member in the form of a tethering member 25. The tethering member 25 may include, but is not limited to, a suture, wire, or nylon cord. The tethering member 25 may enable the user to manipulate or retrieve the sponge 20 once delivered inside of a body structure.

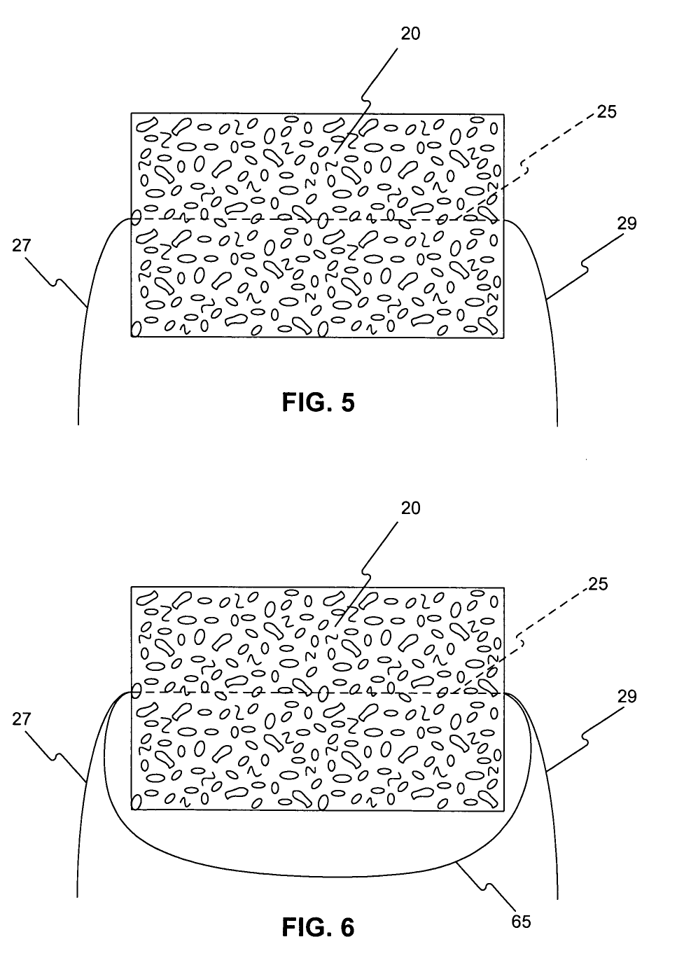

As illustrated in FIG. 5, the tethering member 25 may pass through the body of the sponge 20 and may thus be secured thereto. The tethering member 25 may be inserted through the body of the sponge 20 by using a needle or by other insertion methods or structures known in the art. For example, a hole (not shown) may be punched through the body of the sponge 20 before inserting the tethering member 25. As noted above, the tethering member 25 may have a first leg 27 and a second leg 29, and that the first and second legs 27, 29 may be controlled by the user of the device 5 to manipulate the sponge 20.

As shown in FIG. 6, the tethering member 25 may pass through the body of the sponge 20 twice so as to form a loop 65. As described above, the tethering member 25 may be inserted through the body of the sponge 20, and the loop 65 may thus be formed, by using a needle or by punching a hole through the sponge 20. Passing the tethering member 25 through the sponge 20 twice may improve the user's ability to manipulate or remove the sponge 20.

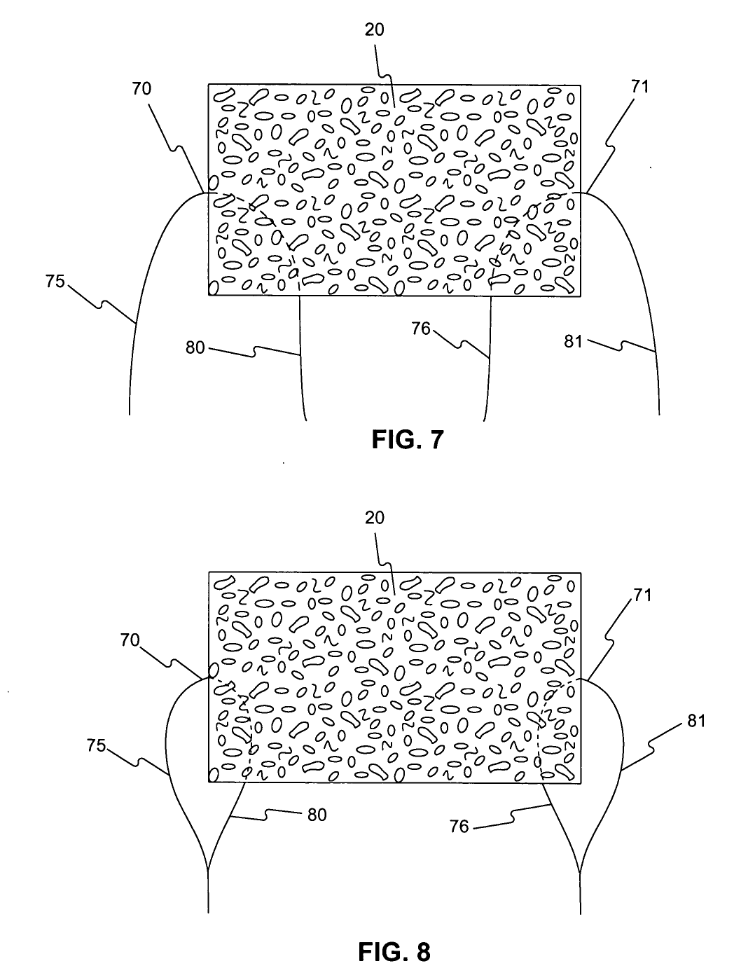

FIG. 7 illustrates an embodiment of the present disclosure in which a first and second tethering member 70, 71 may be used. In such an embodiment, the first and second tethering members 70, 71 may each pass through at least a portion of the sponge 20 and may be inserted thereto as described above. It is understood that the first tethering member 70 may have a first and second leg 75, 80 and that the second tethering member 71 may also have a first and second leg 76, 81.

As shown in FIG. 8, however, the first and second legs 75, 80 of the first tethering member 70, and the first and second legs 76, 81 of the second tethering member 71 may be crimped, knotted, or other wise attached to each other such that each of the tethering members 70, 71 may be controlled by manipulating a single leg. Although not shown, it is understood that greater than two tethering members 70, 71 may be used to manipulate the sponge 20.

Referring back to FIG. 1, the first and second legs 27, 29 of the tethering member 25 may travel external to the device 5 and may enter the delivery tube 10 through a tethering member orifice 85. In some embodiments, the delivery tube 10 may include more than one tethering orifice 85, arranged circumferentially around the distal end 50 of the tube 10 with one or more of the legs 27, 29 extending therethrough. The first and second legs 27, 29 may then exit the distal end 50 of the delivery tube 10. As will be described below, this path of travel may assist in removing the sponge 20. Alternatively, the first and second legs 27, 29 of the tethering member 25 may travel internal to the delivery tube 10, along the length of the tube 10, until exiting the distal end 50 of the delivery tube 10 when the sponge 20 is deployed.

The device 5 of the present disclosure may be used to retrieve stones and other foreign matter located in the bladder, ureter, kidney or other body structure, and may be used in an environment that is relatively fluid filled, or that is relatively dry. The device 5 may be inserted through the urethra of the patient, or alternatively, the device 5 may be inserted percutaneously. It is understood that the device 5 may be used in any location of the body in which a passageway or orifice is being at least partially blocked by a foreign object.

The device 5 may be advanced to a treatment site in a number of different ways. For example, the device 5 may be advanced to the treatment site over a guidewire passing through all or a part of channel 30. This treatment site may correspond to the vicinity of the stone 40 to be removed. The device 5 may also be advanced to the treatment site through an access sheath, or other access or dilatation device known in the art.

Alternatively, the device 5 may be fed to the treatment site fluoroscopically, and may travel through the body without the use of a guidewire or access sheath. To facilitate fluoroscopic feeding the delivery tube 10 may include radiopaque marker bands (not shown) detectable by x-ray. Thus, the user may utilize the urging member 15 to guide the delivery tube 10 to the treatment site and may monitor the position of the device 5 and movement thereof through the use of an x-ray monitor.

As yet another alternative, the device 5 may be used in conjunction with an endoscope (not shown), or other type of intracorporeal scope known in the art. The endoscope may travel through the body to the treatment site in any conventional manner. Once the endoscope is positioned adjacent to the treatment site, the delivery tube 10 may be fed to through an access port of the endoscope to gain access to the stone 40.

As illustrated in FIGS. 2-4, to deliver the sponge 20, the delivery tube 10 may be placed distal the stone 40. The urging member 15 may then be held in place while the delivery tube 10 is retracted. The relative movement of the urging member 15 and the delivery tube 10 serves to deliver the sponge 20 distal the stone 40 in the approximate location of the urging member 15. Alternatively, the delivery tube 10 may be placed distal the stone 40 and held in place while the urging member 15 is advanced, thereby urging the sponge 20 distal the stone 40. This alternative, however, may not deliver the sponge 20 as precisely as the first method described above.

The hydrated sponge 20 may serve several purposes once it has been deployed. For example, the sponge 20 may be used to sweep a stone 40. As described above, and as illustrated in FIG. 4, when placed into a liquid environment, the overall size of the sponge 20 may increase substantially due to fluid absorption. In its expanded state the sponge 20 may expand to conform to the section of the body structure into which it was delivered. The expanded sponge 20 may be soft enough not to damage the surrounding body tissue 45 while expanding, being manipulated within, or being removed from the body structure. In this state, the sponge 20 may also be rigid enough to sweep a stone 40 through the body structure for removal.

To sweep a stone 40 from the body, the user may position the sponge 20 distal the stone 40. The sponge 40 may begin to absorb body fluid and may expand to fit the body structure. Once expanded, the user may pull the sponge 20 in a direction proximal the stone 40 by manipulating the first and second legs 27, 29 of the tethering member 25. As used herein, the term “proximal” means closest to the user. The sponge 20 may then come into contact with a free-floating stone 40, and urge the stone 40 in a proximal direction. Alternatively, if the stone 40 is impacted within the body tissue 45, contact with the sponge 20 may dislodge the stone 40 and urge the stone 40 in the proximal direction. In this way, the sponge 20 may urge the stone 40 through the urinary tract until the stone reaches an exit of the body or a location that otherwise facilitates the removal of the stone from the body. This location may be the bladder or some other body structure allowing for the natural removal of the stone from the body. It is understood that if the sponge 20 contacts a small or sharp stone 40, the stone 40 may become lodged within the surface of the sponge 20. The stone 40 may then be carried with the sponge 20 as the sponge 20 travels in the proximal direction. Also, the sponge 20 may optionally be used to pin the stone 40 between the sponge 20 and the distal end of the delivery tube 10. In such a situation, the delivery tube 10, the sponge 20, and the stone 40 may be removed as one unit if the stone 40 cannot be made to re-enter the delivery tube 10 with the sponge 20.

During the sweeping process, the user may wish to release the stone 40. For example, the stone 40 may encounter a restriction in the body tissue 45 while being swept through the urinary tract. Such a restriction might include, but is not limited to, swelling, scar tissue, or other stones or foreign matter. The restriction may be sized or positioned so as to impede the stone's 40 progress through the urinary tract, thereby necessitating releasing the stone 40 from the sponge 20.

To release the stone 40, the delivery tube 10 may be tracked over the first and second legs 27, 29 of the tethering member 25 until it is positioned distal the stone 40 and the restriction. The user may then pull on at least one of the first and second legs 27, 29 of the tethering member 25 to draw the sponge 20 into the distal end 50 of the delivery tube 10. The sponge 20 may release at least some of the absorbed fluid before entering the delivery tube 10. In some embodiments, a re-capture tube (not shown) of the device 5 may have a larger diameter than the delivery tube 10 to facilitate the re-capture of the expanded sponge 20. The device 5 may then be retracted proximal the stone 40. It is understood that the stone 40 may also be released by manipulating the tethering member 25 so as to discontinue contact between the sponge 20 and the stone 40.

In addition to assisting in stone removal, the device 5 may also assist in immobilizing a stone 40 during stone reduction processes. Such processes may include laser lithotripsy, pneumatic stone reduction, or other processes known in the art. As illustrated in FIG. 9, these processes may involve the use of a laser fiber 35, an air delivery device (not shown), or another device known in the art, to reduce the size of the stone 40 before it is removed from the body.

For example, as described above, the sponge 20 may be located distal a stone 40 so as to prevent small particles of the stone 40 from migrating or escaping from the treatment site during or after a laser fiber 35 or other device acts to break up the stone 40. It is understood that the sponge 20 may be more resistant to damage from the laser fiber 35 than conventional wire baskets or stone retrieval devices. In addition, using a first and second tethering member 70, 71 may increase the dependability of the device 5. For example, if the first tethering member 70 is accidentally severed during laser lithotripsy, the user may still be able to retrieve the sponge 20 using the second tethering member 71.

As shown in FIG. 9, the urging member 15 may be removed from the delivery tube 10 once the sponge 20 has been deployed, and the laser fiber 35 may be fed to the stone 40 through the delivery tube 10. Alternatively, the laser fiber 35 may be fed through the access port of an endoscope, external to the delivery tube 10, while the delivery tube is disposed within the access port. As yet another alternative, the laser fiber 35 may be fed through a different access port of the endoscope than the delivery tube. It is understood that the laser fiber 35 may be activated and otherwise controlled by the user while it is within the body of the patient, and that a proximal end of the laser fiber 35 may connect to a power source (not shown).

Other embodiments of the invention will be apparent to those skilled in the art from consideration of the specification and practice of the invention disclosed herein. For example, the delivery tube 10 may include more than one tethering member orifice 85 and more than one channel 30, and the device 5 may include more than one sponge 20. Alternatively, the device 5 may utilize a web in conjunction with or instead of the sponge 20.

In addition, the device 5 may also include a handle or an actuator operatively attached to the delivery tube 10. Moreover, the at least one tethering member 25 may be attached to the sponge 20 with adhesives such as, for example, waterproof glue. It is intended that the specification and examples be considered as exemplary only, with a true scope and spirit of the invention being indicated by the following claims.

What is claimed is:

1. A medical device, comprising:
a delivery member; and
a sponge configured for deployment from the delivery member and into the urinary tract for assisting in the removal of a stone.

2. The device of claim 1, further including an urging member configured to deploy the sponge from the delivery member.

3. The device of claim 2, wherein the urging member is disposed within the delivery member and proximal the sponge.

4. The device of claim 3, wherein the urging member is removable from the delivery member.

5. The device of claim 1, wherein the sponge is sized to substantially conform to the part of the body into which it is deployed upon absorbing fluid.

6. The device of claim 1, wherein the sponge has a diameter in the range of about 2 mm to about 10 mm in its expanded state.

7. The device of claim 1, wherein the sponge has a compression ratio of about 10 to 1.

8. The device of claim 1, wherein the sponge is secured to the device by at least one coupling member.

9. The device of claim 8, wherein the at least one coupling member includes at least one tethering member connected to the sponge.

10. The device of claim 9, wherein the at least one coupling member includes a plurality of tethering members connected to the sponge.

11. The device of claim 8, wherein at least a portion of the at least one coupling member is disposed within the delivery member.

12. The device of claim 1, further including a laser fiber disposed within the delivery member.

13. A method for assisting the removal of a stone from a body, comprising:
deploying a sponge into the body; and
contacting the stone with the sponge to facilitate removal of the stone from the body.

14. The method of claim 13, wherein the deploying includes deploying the sponge from a tubular member located in the body.

15. The method of claim 14, wherein the sponge is deployed from the tubular member by relative movement of one or both of an urging member and the tubular member.

16. The method of claim 13, wherein the deploying includes locating the sponge distal the stone.

17. The method of claim 13, wherein the contacting includes manipulating at least one coupling member connected to the sponge.

18. The method of claim 13, wherein the contacting includes sweeping the stone to a location allowing for the natural removal of the stone.

19. The method of claim 13, further including capturing the stone within the sponge.

20. The method of claim 13, further including reducing the size of the stone.

21. The method of claim 13, further including removing the sponge from the body.

22. The method of claim 13, wherein the deploying includes deploying the sponge from a tubular member located in the body, and further including drawing the sponge at least partially back into the tubular member.

23. The method of claim 14, wherein the sponge is expanded upon being deployed from the tubular member and is unexpanded when located in the tubular member prior to being deployed.

24. A method for assisting the removal of a stone from a body, comprising:
deploying a sponge into the body; and
substantially prohibiting the stone from migrating distal the sponge while the stone is subject to a procedure.

25. The method of claim 24, wherein the sponge expands to substantially conform to the part of the body into which it is deployed.

26. The method of claim 24, wherein the procedure includes reducing the size of the stone.

27. The method of claim 26, wherein the procedure includes using a laser fiber to assist in the reducing the size of the stone.

28. The method of claim 24, further including capturing the stone in the sponge.

29. The method of claim 24, further including sweeping the stone.

30. The method of claim 24, further including controlling the sponge with at least one coupling member connected to the sponge.

31. The method of claim 24, further including removing the sponge from the body.
